(19) United States
(12) Patent Application Publication
(10) Pub. No.: US 2008/0247947 A1
(43) Pub. Date: Oct. 9, 2008

(54) High dose 111In-pentetreotide therapy of neuroendocrine tumors

(76) Inventor: Ebrahim S. Delpassand, Houston, TX (US)

Correspondence Address: OSHA LIANG L.L.P., 1221 MCKINNEY STREET, SUITE 2800, HOUSTON, TX, 77010, US

(21) Appl. No.: 11/784,251

(22) Filed: Apr. 6, 2007

(51) Int. Cl. A61K 51/08

(52) U.S. Cl. 424/1.69

(57) ABSTRACT

A method for treating a neuroendocrine tumor is disclosed, which includes administering a first dose of ¹¹¹In-pentetreotide to a subject having the neuroendocrine tumor; and administering a second dose of ¹¹¹In-pentetreotide to the subject after a selected duration has elapsed after the administering of the first dose, wherein the first dose and the second dose are each about 400 mCi or higher, and wherein the selected duration is at least a week. In another method, the first dose and the second dose are each about 500 mCi and the selected duration is between 10 and 12 weeks. The method may further include administration of a third dose. A composition for treating a neuroendocrine tumor includes ¹¹¹In-pentetreotide having a radioactivity of about 400 mCi or higher in a single dose.

### BACKGROUND OF INVENTION

1. Field of the Invention

This invention relates to methods for treating neuroendocrine tumors. In particular, this invention relates to treatment of neuroendocrine tumors with targeted radiotherapy.

2. Background Art

Neuroendocrine tumors (NETs) are a heterogeneous group of neoplasms that originate from the neural, endocrine, cardiac, osseous and gastrointestinal tissues. NETs arising from the gastrointestinal tract, the lungs, and thymus are often referred to as carcinoid tumors (CT). Depending on their origins, NETs may or may not share common histological, metabolic and ultrastructural features. For example, NETs of lung origin are poorly differentiated and highly aggressive, while most other NETs are generally slower growing.

NETs retain multi-potent differentiation capacities and can produce and secrete a variety of metabolically active substances, including amines, peptides, tachykinins and prostaglandins. Systemic release of one or more of these compounds contributes to the high morbidity of these diseases. NETs may induce one or more of following symptoms: neurologic (pain, motor or sensory losses), psychologic (such as depression or psychosis), cardiovascular (palpitation, hypertension and heart failure), respiratory (shortness of breath), gastrointestinal (diarrhea, peptic ulcers and abdominal pain), endocrinological (diabetes and acromegaly) and dermatological (areas of flushing), along with abnormalities in various forms such as: Cushing's syndrome, Carcinoid, Zollinger-Ellison, insulinema, VIPoma, glucagonoma, somatostatinoma, GRFoma, CRFoma, PPoma, and neurotensinima. Because symptoms of NETs are usually vague, nonspecific and vary from organ to organ, NETs are not easily diagnosed and many NETs are found only during surgeries for other disorders, such as appendicitis, pancreatitis or small bowel obstruction. The average time from symptom onset to the diagnosis of NETs is more than 9 years. Due to the indolent nature of NETs, most patients do not seek treatments until they have metastatic diseases. As a result, their prognosis is poor.

NETs are rare, accounting for only 0.5% of all malignancies. The overall prevalence of NETs in the United States is estimated to be 2 cases per 100,000 individuals. Thus, NETs meet the FDA's criteria for orphan disease status. In the last decade, incidence of NETs has been rising due to increased awareness and improved diagnostic tools. NETs are more common in men than in women (10.6 vs. 5.6 per million people) and more common in blacks than in whites (9.2 vs. 5.4 million people, respectively).

The primary sites for NETs are in the gastrointestinal tract (62%), especially in the appendix and small bowel. NETs account for 13-34% of all tumors of the small bowel and 17-46% of all malignant tumors of the small bowel. Tumors of the lung rank second as the source for NETs, accounting for 23%. Survival is mainly dependent on disease stages, with a five year survival rate of 93% for local diseases, 74% in regional diseases, and only 19% for patients with metastatic diseases. Due to slow progression of NETs, patients live longer than those with more serious and fast growing cancers, and suffer from long lasting symptoms with tremendous socioeconomic loss. This creates a high morbidity for NETs and demands more effective treatment methods.

Currently, treatment options for NETs are limited. Surgery is the principal therapy for benign or localized NETs. However, surgery is less useful in advanced NETs. Conventional cytotoxic cancer therapies have a low efficacy (<15%) in NETs and are often associated with severe dose-limiting toxicities. Single agent or combination chemotherapy regimens for management of NETs have not been promising. Treatment with various chemotherapeutic agents have shown only minimal efficacy against NETs, with response rates (based on either tumor regression or decrease in urinary hormone secretion) ranging from 0 to 31% and durations of responses generally less than one year. Trials with combination chemotherapy have failed to demonstrate increased response rates. For example, two-drug combinations of streptozotoxin and 5-fluoruracil, streptozotocin and cyclophosphamide, streptozotocin and doxorubicin, etopodise and cisplatin, dacarbazine and 5-flurouracil, and CCNU and 5-fluoruracil exhibit no significant improvement over single-agent therapies and are associated with significantly increased toxicities.

Furthermore, external-beam radiation therapy has not shown any efficacy in controlling local NETs. Interferon (IFN) therapy has been used in protocol-based management of carcinoid tumors with reported biochemical and tumor response rates of 40% and 12%, respectively. However, IFN therapy is associated with significant toxicity, including fever, anorexia, weight loss, fatigue and myelosuppression. Due to the high incidence of toxicities and the low tumor response rates, routine use of IFN in the treatment of NETs is limited.

A major problem with conventional cytotoxic cancer therapies lies in their lack of specificity and severe dose-limiting toxicities. A potential solution is to target cancer cells to limit systemic exposure and side effects of a therapeutic agent. Targeted therapy can be based on any unique features that are cancer cell specific. For example, NETs are found to have more abundant expression of somatostatin receptors, as compared with normal cells. Therefore, reagents that specifically target the somatostatin receptors can be promising therapeutics for the treatment of NETs.

Somatostatin is a peptide hormone that regulates the endocrine system and affects neurotransmission and cell proliferation. Somatostatin has two active forms produced by alternative cleavage of a single preproprotein: a 14 amino acid peptide and a 28 amino acid peptide. Somatostatin is secreted by the hypothalamus, stomach, intestine, and the delta cells of the pancreas. Somatostatin binds to somatostatin receptors and inhibits the release of numerous secondary hormones.

Somatostatin receptors (which have five subtypes, sst1-5) are membrane-bound G-protein coupled receptors and are expressed on more than 80% of carcinoid tumors. High densities of somatostatin receptors are found on NETs of the gastoenteropancreatic axis (such as insulinomas, gastinoma, glucagonomas, lymphomas and nonfunctional islet cell tumors) and neural crest tumors (such as pheochromocytoma, medullary carcinoma of the thyroid and small cell carcinoma of the lungs).

Binding of somatostatin to the somatostatin receptors exerts a cytostatic effect on tumor cells and inhibits symptoms of carcinoid syndrome, such as flushing and diarrhea. However, the clinical utility of somatostatin is limited by its short in vivo half-life (2-4 minutes). The introduction of somatostatin analogues with longer half-lives has overcome the drawbacks of native somatostatin. Octretotide is an 8 amino acid analogue of somatostatin that binds to sst2 or sst5 positive tumors and induces physiological effects similar to the native somatostatin. With an in vivo half-life of 90-120 minutes, octreotide has been shown to be effective in controlling the symptoms of carcinoid syndrome. In addition, some clinical studies suggest that octreotide therapy can result in tumor stabilization in 50-55% of patients with advanced disease, but it is unclear if this stabilization is associated with a significant increase in survival.

Traditionally, in situ radiotherapy has been accomplished with high-energy radioactive compounds such as ¹¹¹In-labelled monoclonal antibodies. Unfortunately, these compounds have shown limited success due to their large size and their propensity to induce immune responses. Smaller peptide analogues of somatostatin offer an alternative to monoclonal antibodies for systemic “targeted” radiotherapy. Encouraging results have been obtained with octreotide coupled to Yttrium-90, a high-energy beta particle emitter, with phase II trials showing radiological response rates in up to 23% of patients with metastatic NETs. However, the use of Yttrium-90 agents is limited by severe renal and hematological toxicities.

Scintigraphy with ¹¹¹In-pentetreotide (a diethylene triamine pentaacetic acid (DTPA) conjugate of octreotide, commercially known as Octreoscan®, available from Mallinckrodt, Inc., St. Louis, Mo.) is frequently used to localize previously undetected primary or metastatic NETs. ¹¹¹In emits γ photons of two energies (172 and 245 keV), which are useful in scintigraphy. ¹¹¹In also emits Auger and conversion electrons with tissue penetration of 0.02-10 μm and 200-500 μm, respectively. These Auger and conversion electrons from ¹¹¹IN can potentially be used in radiotherapy.

At doses (up to 180 mCi) higher than those used in scintigraphy, ¹¹¹In-pentretotide has been evaluated as a therapeutic agent and showed some potential. Although the exact cytotoxic mechanism of ¹¹¹In at the cellular level is unknown, studies have shown that after internalization through somatostatin receptors expressed on the cellular and nuclear membranes, ¹¹¹In is translocated to the perinuclear area and into the nucleus of tumor cells. Although it possesses a short irradiation range, it is assumed that the nuclear localization allows DNA strand breakage by Auger electrons, resulting in the tumoricidal effects of ¹¹¹ In.

Pentetreotide is a DTPA-conjugate of octreotide. ¹¹¹In-pentetreotide binds to somatostatin receptors on cell surfaces throughout the body. Within an hour of injection, a majority of the tracer activity distributes from plasma to extra-vascular body tissues and concentrates in tumors containing a high density of somatostatin receptors. After background clearance, somatostatin receptor-rich tissue may be imaged. In addition to somatostatin receptor-rich tumors, normal pituitary gland, thyroid gland, liver, spleen and urinary bladder also are visualized in most patients, as is the bowel, to a lesser extent.

The first clinical trial of ¹¹¹In-pentretotide for treatment of NETs was performed by Krenning et al. in the Netherlands in 1994. Preliminary data from this study demonstrated the safety of repeated doses of 180 mCi ¹¹¹In-pentetreotide administered every 3 weeks for 10 occasions. In this initial study, responses to ¹¹¹In-pentetreotide treatment correlated with receptor expression. Subsequent studies conducted by Krenning et al. confirmed these findings.

Likewise, other researchers have reported significant responses to high-dose ¹¹¹In-pentretotide therapy with limited toxicity. These studies, performed mostly in Europe and summarized in FIG. 1, vary in patient selection, dosing, total activity administered and reported outcomes, but all show significant benefits and safety of ¹¹¹In-pentetreotide therapy. These results demonstrate that ¹¹¹In-pentetreotide therapy is safe and can significantly defer the occurrence of fatal and nonfatal events in patients that fail to respond to conventional treatment.

However, the long term efficacy of this treatment in patients with disseminated NETs is questionable. A 2003 study of 16 patients given repeated doses of ¹¹¹In-pentretotide (81-134 mCi), showed that while up to 70% had some short term benefits, only 31% of patients had sustained benefit 18 months after the treatments.

### SUMMARY OF INVENTION

One aspect of the invention relates to methods for treating a neuroendocrine tumor. A method in accordance with one embodiment of the invention includes administering a first dose of ¹¹¹In-pentetreotide to a subject having the neuroendocrine tumor; and administering a second dose of ¹¹¹In-pentetreotide to the subject after a selected duration has elapsed after the administering of the first dose, wherein the first dose and the second dose are each about 400 mCi or higher, and wherein the selected duration is at least a week. In preferred methods, the first and the second doses are each about 500 mCi and the selected duration is bout 6-16 weeks, more preferably about 10 to 12 weeks. Some methods of the invention may further include administering a third or more doses of ¹¹¹In-pentetreotide to the subject.

Another aspect of the invention relates to compositions for treating a neuroendocrine tumor. A composition in accordance with one embodiment of the invention includes ¹¹¹In-pentetreotide having a radioactivity of about 400 mCi or higher in a single dose.

Other aspects and advantages of the invention will be apparent from the following description and the appended claims.

### BRIEF DESCRIPTION OF DRAWINGS

FIG. 1 shows a table summarizing results from prior treatments of NETs with ¹¹¹In-pentetreotide.

FIG. 2 shows a table summarizing the low toxicities induced by a method of treating patients having NETs in accordance with one embodiment of the invention.

FIG. 3 shows a chart illustrating the efficacy of a method of treating patients having NETs in accordance with one embodiment of the invention.

FIG. 4 shows a flow chart illustrating a method for treating patients having NETs in accordance with one embodiment of the invention.

FIG. 5 shows a schematic illustrating a general scheme of methods in accordance with embodiments of the invention.

FIG. 6 shows tumor images from a patient before and after the treatment in accordance with one embodiment of the invention.

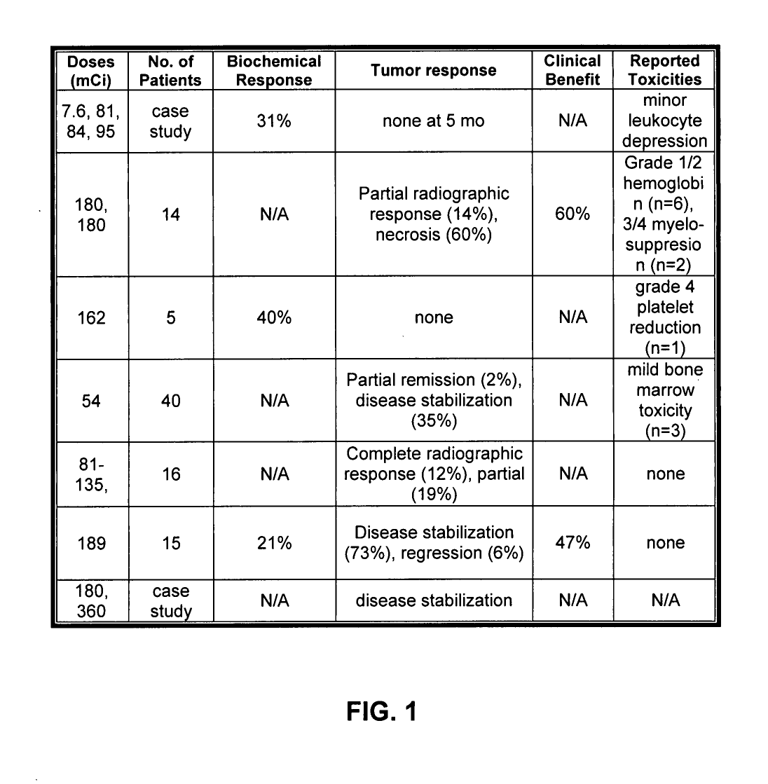

### DETAILED DESCRIPTION

Embodiments of the invention relate to methods for treating neuroendocrine tumors, using targeted radiation. In particular, methods in accordance with embodiments of the invention use high-dose ¹¹¹In-pentetreotide to target neuroendocrine tumor cells. As used herein, “high-dose” of ¹¹¹In-pentetreotide means high radiation doses, not high peptide concentrations. Furthermore, the “high-dose” radiation of ¹¹¹In-pentetreotide refers to about 400 mCi per dose or higher, preferably about 500 mCi per dose or higher.

Neuroendocrine tumors originate from the hormone-producing cells of the neuroendocrine system. The neuroendocrine system includes cells that are a cross between traditional endocrine, or hormone-producing cells, and nerve cells. Neuroendocrine cells are found throughout the body, including organs, such as the lungs and gastrointestinal tract. Methods of the invention may be applied to neuroendocrine tumors that are local or metastatic.

As noted above, ¹¹¹In-pentetreotide at doses up to 180 mCi have been investigated as treatment for NETs. However, while short term benefits are reported for most patients, long term effects are questionable. Because both Auger and conversion electrons from ¹¹¹In have short tissue penetration ranges, such short-lived responses may be attributable to incomplete eradication of the tumor cells, leading to the survival of resistant clones. Over time, surviving clones will repair the damages that might have been induced by the exposure to ¹¹¹In radiation. After a “recovery” period, such surviving clones may start to expand, resulting in the recurrence of the tumors. Based on this hypothesis, methods in accordance with embodiments of the invention are designed to minimize potential survivors after each treatment and to repeat the treatments with a selected period between the treatments to maximize the effectiveness in preventing the re-growth of any surviving clones.

Therefore, in accordance with embodiments of the invention, multiple cycles of high dose (e.g., 500 mCi) ¹¹¹In-pentetreotide (trade name: NeuroendoMedix®, IND 72,037) are used to deliver targeted radiation to the DNA of tumor cells by internalization of the labeled compound through somatostatin receptors on the surface of NETs. Specifically, methods of the invention use high radiation doses (e.g., 500 mCi) in each treatment to minimize the formation of surviving (or resistant) clones and use repeated treatments with intervals of several days or weeks to maximize the overall treatment efficacy. In accordance with embodiments of the invention, the repeated treatments are spaced by several days to several weeks in recognition of the slow-growing characteristics of the NETs. Methods of the invention increase treatment efficacy, while at the same time this approach limits systemic side effects.

### EXAMPLES

In a non-randomized trial, 32 patients (21 men and 11 women, from 16-83 years old (average 56 years old)) were tested. Among these patients, there were 25 Caucasions (78%), 2 Blacks (6%), 4 Hispanics (12.5%) and 1 other (3%). All patients had progressive neuroendocrine tumors (21 Carcinoma, 7 Islet Cell Carcinoma of the Pancreas, 1 Pituitary Adenoma, 1 Glucagonoma, and 1 Pheochromocytoma) and all had failed first line therapy (23 chemotherapy, 12 radiotherapy, and 28 surgery).

These patients had been diagnosed as having neuroendocrine tumors using serum chromogranin A (CgA) as a main tumor marker, and by somatostatin receptor scintigraphy for tumor localization. Other markers used include serum serotonin, pacreastatin, gastrin, normetanephrine, alkaline phosphatase (ALP), and 24-hour urine 5-HIAA. Histopathological examination also was used as a confirmatory test. Comparison of parameters was done between the amount of each marker before and after treatment All patients were in a progressive state and had received and failed standard therapy before entering the study.

Fourteen patients received one therapy (ranging from 473.5 to 511.6 mCi, with an average of 489.2 mCi) and 18 patients received two treatments with the cumulative doses ranging from 955.1 to 1,006.1 mCi (average=982.8 mCi/patient). Patients were evaluated for evidence of hematological, renal and hepatic toxicities using NCI common toxicities criteria and followed for an average of 9 months (ranging from 1.7 to 17.2 months).

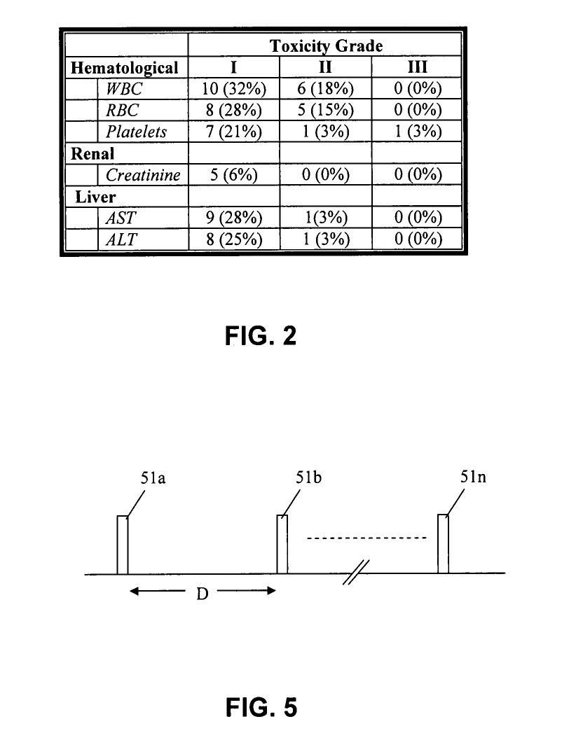

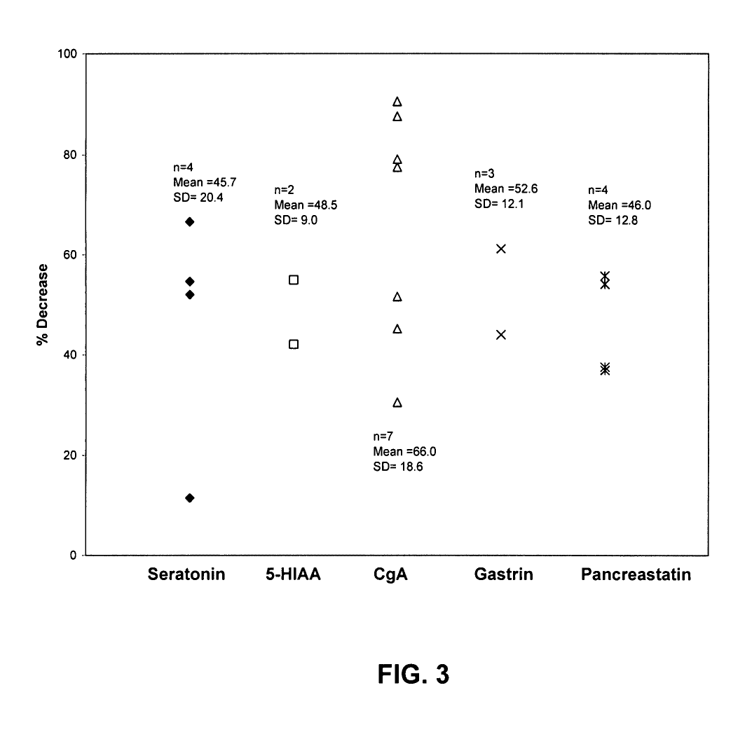

Among these patients, seventeen (53%) had grade I or II hematological toxicities, while one patient had grade III thrombocytopenia with nadir mean of 5 weeks (ranging from 4 to 6 weeks) after therapy (FIG. 2). These toxicities were transient, lasting from 1 to 6 weeks (averaging 3 weeks). None of the patients required supportive therapy. One patient had grad II liver toxicity which appeared 4 weeks after the therapy and resolved in week 5. There was no evidence of renal toxicity during the follow-up period. Among the 18 patients (13 men and 5 women, from 41 to 83 years old (average age 59)), who were treated with two cycles of high dose (500 mCi) ¹¹¹In-pentetreotide, therapeutic efficacy of this treatment regimen is evident (FIG. 3). These patient had progressive neuroendocrine tumors and failed first-line therapy. According to the pre-therapy diagnostic ¹¹¹In-pentetreotide scan, these patients had grade 3 to grade 5 tumors. In this trial, each dose of ¹¹¹In-pentetreotide was infused over a period of 3 to 6 hours. The two treatments were spaced a few weeks (e.g., 3-6 weeks) apart. The patients were monitored for clinical, metabolic, and imaging responses based on using Response Evaluation Criteria in Solid Tumors (RECIST) criteria (Therasse P, Arbuck S G, Eisenhauer E A, et al. New guidelines to evaluate the response to treatment in solid tumors. European Organization for Research and Treatment of Cancer, National Cancer Institute of the United States, National Cancer Institute of Canada. Journal of the National Cancer Institute 2000; 92(3):205-16.).

After the treatments, sixteen out of the 18 patients achieved stable disease by imaging criteria. Metabolic responses (defined as 25% or more decline in chromogranin or other markers) are achieved in 72% of the patients. Clinical responses (as defined by functional living index (FLI) questionnaire and physical examination) were achieved in 84% of the patients. These results indicate that the repeated treatments are really effective.

Side effects from the double-dose treatments were minimal. Ten patients (55%) had grade I or grade II hematological toxicities and one patient had grade III thrombocytopenia, with a nadir mean of 5 weeks after therapy. These hematological toxicities were found to last for 1-6 weeks. None of the patients required supportive therapy. One patient had grade II liver toxicities which appeared 4 weeks after the therapy and resolved on week 5. No renal toxicity was observed during the follow-up period, ranging from 3.93 to 17.23 months (average=9.87 months). These results suggest that there was no significant cumulative toxicity effects with the repeated treatments.

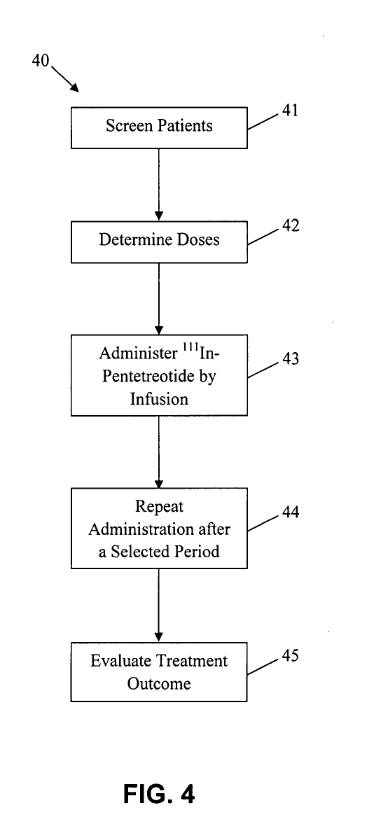

FIG. 4 outlines a general method 40 in accordance with one embodiment of the invention. As shown, the treatment typically starts with screening for the proper patients who might benefit form the treatments (step 41). Then, based on various factors (disease stage, patient age, body weight, etc.), a proper dose of ¹¹¹In-pentetreotide for an individual in need of such treatment may be determined (step 42). In general, a high dose (e.g., 500 mCi) if preferred. A method for determining the proper dose will be described in detail below. Next, the dose is administered to the patient (step 43). In general, the administration is via infusion over a period of several hours (e.g., 3-5 hours) because it has been shown that infusion over several hours enhances drug accumulation via receptor-mediated endocytosis. See U.S. Pat. Nos. 6,180,082 and 6,630,123 issued to Woltering et al.

After a selected period of time, which may be a few days to a few weeks, the administration is repeated (step 44). Again, the administration of ¹¹¹In-pentetreotide is preferably via infusion over a few hours. If necessary, the treatment may be repeated for a desired number of times, i.e., step 44 may be repeated. Note that each administration of ¹¹¹In-pentetreotide may or may not involve the same dose. In general, high doses (e.g., 500 mCi) for each treatment are preferred.

FIG. 5 illustrates a schematic depicting the treatment regimen that involves repeated administration of ¹¹¹In-pentetreotide. As shown in FIG. 5, the treatments 51a, 51b, . . . 51n are spaced apart in time. Note that the intervals D need not be identical for each successive treatment. As noted above, each treatment is preferably performed via infusion over several hours. The intervals D between treatments preferably are several days to several weeks because NETs are slow-growing tumors and any surviving clones will take time to grow.

Referring to FIG. 4 again, while the treatment is ongoing or after the administration of ¹¹¹In-pentetreotide, the patients may be evaluated for treatment efficacy and/or possible toxicities (step 45). Any suitable protocols known in the art may be used for such evaluations, and some examples are described in detail below.

Note that the methods shown in FIGS. 4 and 5 are for illustration only. One or ordinary skill in the art would appreciate that modifications of these methods are possible without departing from the scope of the invention. For example, the patients may be pre-screened or referred by the physicians, and therefore the screen step 41 may be skipped. Similarly, it may not be necessary to determine a specific doses for each individual patients. Although the follow up and evaluation are desirable, these procedures are not necessary for the treatment (or cure) of the disease.

The following describe methods and examples for making and using the high-dose ¹¹¹In-pentreotide. One of ordinary skill in the art would appreciate that specific numbers and service providers referred to in these examples are for illustration only, and not intended to limited the scope of the invention. Furthermore, other suitable methods and/or procedures known in the art may also be used instead of those specifically described.

### Purification of ¹¹¹InCl₃

Indium-111 chloride (¹¹¹InCl₃) was purchased from MDS Nordion (Ottawa, ON, Canada). Because methods of the invention require high doses of radioactive ¹¹¹In, special care was taken to prevent reduced radiolabeling efficiency due to the presence of cadmium and other impurities (such as iron, lead, zinc, aluminum and copper) commonly found in ¹¹¹InCl₃ solution. Therefore, in collaboration with IsoTex Diagnostics, Inc. (Friendswood, Tex.), an FDA registered facility for the manufacturing of radiopharmaceuticals, the radioactive solution was purified prior to radiolabeling. The radionuclide was purified based on the methodology disclosed in U.S. Pat. No. 6,162,648 issued to Maloney et al., which is incorporated by reference in its entirety, in order to obtain ultra-pure ¹¹¹InCl₃ and ensure satisfactory and reproducible radiolabeling yields. Only highly purified, high specific activity ¹¹¹InCl₃ was used. Each batch of purified ¹¹¹InCl₃ should be analyzed for the total activity, total volume, radionuclide purity and the radionuclide identity. The sterility and non-pyrogenic status of the radiochemical solution should also be confirmed.

### Formulation of Therapeutic Doses of ¹¹¹In-Pentetrotide

The radiolabeling procedures were adapted and modified from the procedures outlined in the package insert that come with Octreoscan® from Mallinckrodt, Inc. (St. Louis, Mo.). These procedures were performed by South Texas Nuclear Pharmacy (Houston, Tex.), where a certified radiopharmacist compounded the therapeutic doses of ¹¹¹In-pentetrotide and provided quality control of the final products.

Through calculations based on molar ratios of the somatostatin analog contained in Octreoscan® kits as used in the diagnostic application, it was determined that a suitable labeling ratio with ¹¹¹InCl₃ was up to 200 mCi per vial of peptide (containing 10 μg of the peptide). As a result, 3 commercial vials of pentetreotide were combined into a single vial prior to addition of the isotope. This was performed in a sequential manner through the addition of approximately 1.3 ml of saline (0.9%) or sterile water to the first vial, gently inverted and rotated to dissolve all material, then withdrawn and applied to each successive vial until reaching the final vial in the sequence. Each vial was inverted and gently tapped to ensure that all dissolved material was withdrawn. The final tagging was performed in the reaction vial provided by the manufacturer. Special care was taken during this process to ensure that the full contents of each vial were transferred at each step.

The synthesis of a therapeutic dose of ¹¹¹In-pentetreotide was obtained by adding large radioactive amounts of ultra-pure ¹¹¹InCl₃ (e.g., 500 mCi) to the peptide-containing vial under aseptic conditions. The resulting solution was gently swirled at 15 minute intervals to ensure mixing and incubated for at least one hour. The solution was inspected to ensure no formation of particulates and then analyzed for quality control.

### Quality Control

Any package contamination was checked by performing a wipe test prior to shipment or upon receipt using a Ludlum® 14C survey meter. All measurements of radioactivity were performed using a dose calibrator (Capintec® CRC-15W) set for the energy peaks of ¹¹¹In. Quality control was performed using a radioactive detector combined with high-performance liquid chromatography (radio-HPLC). After Sep-Pak cartridges purification of the final product, the therapeutic doses described above were found to have suitable radiochemical purity (RCP>95%) for administration to patients. Through preliminary experiments and analysis of the initial patient doses, it was found that radiochemical purities of >98% could be achieved. Additionally, the 500 mCi dose did not adversely affect radiochemical purity or the integrity of the peptide. Percent hydrophilic impurities and non-elutable impurities were also calculated and were typically <1%.

### Dosimetry and Biodistribution of In-111 Octreotide

OctreoScan® scintigraphy was performed for dosimetry, visualization of tumor spread and assessment of radionuclide uptake intensity. In such tests, patients were injected intravenously with a low-dose (e.g., 5 to 6 mCi) [¹¹¹In-DTPA-D-Phe1]-octreotide (Mallinckrodt Medical, St. Luis, Mo.-USA). Static anterior-posterior images were collected, and a SPECT investigation (single photon emission computed tomography) was carried out over the regions of tumor, using a dual detector gamma scintillation SPECT camera equipped with a medium-energy general purpose collimator (Dual-detector Siemens e-cam).

For each patient, whole body images were taken at 45 minutes after injection before void, and at 4, 24, and 48 hours. Regions of interest (ROI) was drawn around the major organs: liver, spleen, kidneys, bladder, femur and whole body (remainder). The data point representing the percentage injected dose (% ID/organ) was then created and fitted to a monoexponential, bioexponential, or an uptake-and-clearance curve. After curve fitting and integration, the cumulative radio activity in each organ and the residence time (t) in each organ were calculated. The femur curve was used to estimate the cumulative activity in bone marrow. The radiation absorbed dose was calculated by entering the corresponding residence time into the OLINDA software program (Stabin M G, Sparks R B, Crowe E. OLINDA/EXM: the second-generation personal computer software for internal dose assessment in nuclear medicine. J. Nucl. Med., 2005; 46(6):1023-7, Vanderbilt University, Nashville, Tenn.), which computes the radiation-absorbed dose values as mGy/MBq or (rad/mCi) for each of the target organ. From the curve fitting, the residence times of ¹¹¹In-pentetreotide in various organ were found to vary from 50 to 55 hours.

### Treatment Protocol

Patients were injected with ¹¹¹In-pentetreotide at an average dose of 489.2 mCi (ranging from 473.5 to 511.6 mCi) as an intravenous infusion in physiological saline using a specially designed IV pole to decrease radiation exposure to the personnel. The infusion took about 3 to 5 hours, in an outpatient setting. The amount of octreotide administered was 30 mg. Patients were evaluated for hematological, renal and hepatic toxicities using NCI common toxicities criteria on week 4, 5, 6, and 7 after treatment, and every three months thereafter. Patients were followed for an average of 8.69 months with a range of 1.7-17.2 months. 18 patients received a second treatment 10-12 weeks after the first therapy with an average cumulative dose of 982.8 mCi (ranging from 955.1 to 1006.1 mCi). These patients were evaluated for a clinical and metabolic responses beginning 4 weeks after their second therapy.

### Safety of ¹¹¹In-Pentetreotide Therapy

There was no evidence of significant treatment associated toxicity following either one or two cycles of ¹¹¹In-pentetreotide therapy (FIG. 2). Seventeen patients (53%) had grade I or II hematological toxicities with a nadir mean of 5 weeks (ranging from 4 to 6 weeks) after the therapy, and one patient had grade III thrombocytopenia. These hematological toxicities lasted for an average of 3 weeks (ranging from 1 to 6 weeks). One patient had grade II liver toxicity, which appeared 4 weeks after the therapy but the symptom resolved in week 5. No significant short or long term renal toxicity was observed up to 17 months after initial therapy and 13 months after the second therapy. None of the patients required supportive therapy. One patient developed nose bleeds 4 weeks after treatment. The patient was treated symptomatically with no need for blood or platelet transfusion.

Two patients died during the course of this study. One patient was a 54 year old white male who died 4 weeks after his first cycle of therapy. This patient had stage IV neuroendocrine disease with 95% involvement of the liver, marked Ascites, retroperitoneal lymph node metastasis, and peritoneal seeding. The treatment goal was mainly palliative to decrease severe diarrhea and abdominal pain. The cause of death was massive tumor burden, cachexia, and cardiac failure. The second patient was a 63 year old white male with stage IV Carcinoid. He had extensive liver, bone and mesenteric involvement by Carcinoid. He had failed prior surgery, chemotherapy, hepatic embolization, and cold Sandostatin therapy. He died 6 weeks after the therapy secondary to tumor burden.

### Efficacy of ¹¹¹In-Pentetreotide Therapy

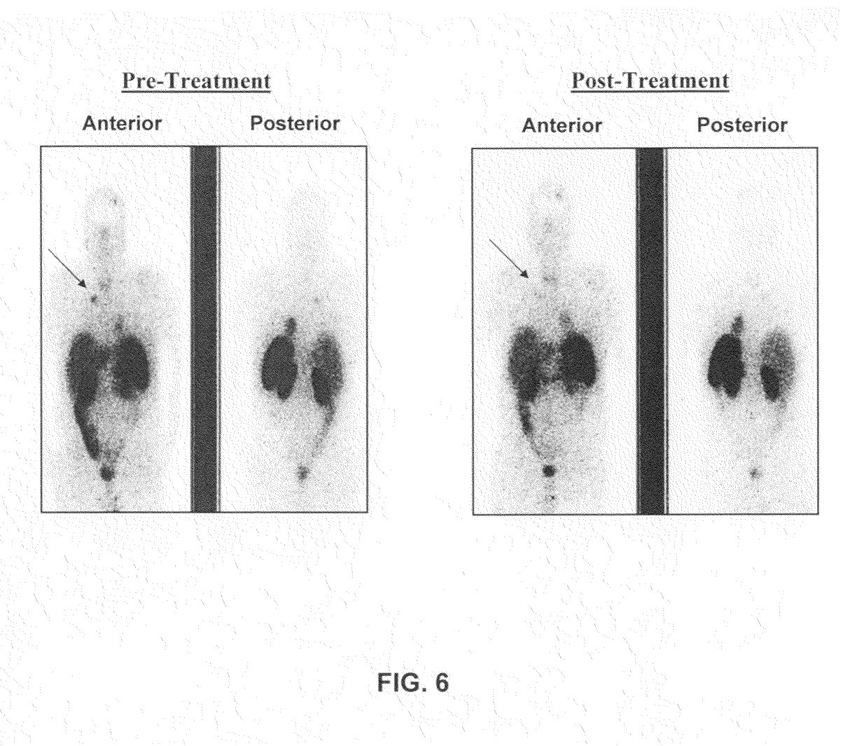

The efficacy of ¹¹¹In-pentetreotide therapy was evaluated in 18 patients who received two cycles of therapy. The primary endpoint was clinical, metabolic and imaging response using RECIS criteria. Sixteen out of 18 patients (88%) achieved stable disease by imaging criteria (a less than 25% decrease in maximum SUV, 50% decrease in the size of any measured lesion, no more than a 25% increase in the size of any measured lesion and no new areas of disease). Metabolic response (defined as 25% or more decline in Chromogranin or other markers) was achieved in 72% of the patients (FIG. 3). Clinical response, defined by Functional Living Index (FLI) questionnaire and physical exam, was achieved in 84% of the patients. Tumor image before and after treatment showing no progression (FIG. 6).

These results suggest that multiple high-doses (e.g., 500 mCi) of ¹¹¹In-pentetreotide therapy are effective in patients with progressive disseminated neuroendocrine tumors. There was no evidence of significant treatment associated toxicity following an average cumulative dose of 982.8 mCi of ¹¹¹In-pentetreotide therapy for an average follow up time of 8.69 months post-treatment. The refore, maximum tolerated dose has not been reached. The safety profile of this regimen suggests that the doses may be escalated or cycles extended non-myeloablative dose escalation.

### Treatment Regimen

Before treatments, patients may have a diagnostic whole body scan with dosimetry to calculate total body residence time using low-dose (e.g., 5-6 mCi) ¹¹¹In-pentreotide. Occupancy factor will be determined from patient's living condition situation (number of persons in contact with the patient, time of contact within 1 meter of patient). Subsequently, the maximum dose (D) to individual from patient will be calculated for an occupancy factor (OF) at 1 meter using measured dose rate at 1 meter in mrem/h and the calculated residence time in hours. Subsequently, the maximum dose (D) to individual patient will be calculated from an occupancy factor (OF) at 1 meter using measured dose rate at 1 meter in mrem/h and the calculated residence time in hours.

Patients will be advised to adhere to specific instructions to minimize exposure to other persons during the periods when they will have high radioactivity in their body. The duration of the requirement to comply with these instructions will be individually determined for each patient as calculated from the absorbed dose (D×Residence time).

For each treatment, patients would receive high dose (e.g., 500 mCi) In-111 pentetreotide via intravenous infusion over 4 to 6 hours. Oral and intravenous hydration over the subsequent 24 hrs will follow. For patients who are not qualified for outpatient release, hospitalization would be required until the residual dose is below a safety level (e.g., 60 mCi) or after a selected period of time (e.g., approximately 48 hours).

After treatments, patients may be monitored or evaluated with various means, such as a scan to confirm targeting of the lesions at 2-4 days after therapy. Patients may also be given a physical exam and physician evaluation 4 weeks after each therapy cycle and every 6 weeks after the third therapy. Laboratory studies for monitoring or evaluation may include CBC, Complete Metabolic Panel, estimate of GFR, 24-hour creatinine clearance and tumor markers (5-HIAA, Chromogranin A, Gastin, Pancreastatin, Seratonin, Calcitonin), which may be performed on weeks 4-8 after each treatment. Radiological evaluation of tumors may be performed using FDG-PET, Octreoscan, CT scan and MRI, for example, at 12 weeks following each therapy cycle.

The above describes examples for methods of the invention. One of ordinary skill in the art would appreciate that various modifications are possible without departing from the scope of the invention. For example, while preferred examples use 500 mCi for each treatment, the useable doses may be adjusted according to the patient conditions. Particularly, the above results indicate that little toxicities are elicited with 500 mCi doses. This fact suggest that doses higher than 500 mCi may also be used. Alternatively, the treatment cycles may be increased to include 3, 4 or more cycles.

Advantages of the present invention may include one or more of the following. Methods of the invention can be used to effectively treat NETs. With the higher doses and repeated treatments, better treatment outcomes an longer lasting effects are observed. With methods of the invention, little toxicities resulted before therapeutic effects are evident.

While the invention has been described with respect to a limited number of embodiments, those skilled in the art, having benefit of this disclosure, will appreciate that other embodiments can be envisioned that do not depart from the scope of the invention as disclosed herein. Accordingly, the scope of the invention shall be limited only by the attached claims.

What is claimed is:

1. A method for treating a neuroendocrine tumor, comprising:
administering a first dose of ¹¹¹In-pentetreotide to a subject having the neuroendocrine tumor; and
administering a second dose of ¹¹¹In-pentetreotide to the subject after a selected duration has elapsed since the administering of the first dose,
wherein the first dose and the second dose are each about 400 mCi or higher, and wherein the selected duration is at least a week.

2. The method of claim 1, wherein the first dose and the second dose are each about 500 mCi.

3. The method of claim 1, wherein the administering the first dose and the administering the second dose are performed by intravenous infusion over a duration of from 3 to 6 hours.

4. The method of claim 1, wherein the selected duration is from 6 to 16 weeks.

5. The method of claim 1, wherein the selected duration is from 10 to 12 weeks.

6. The method of claim 1, further comprising administering a third dose of ¹¹¹In-pentetreotide to the subject after a second duration has elapsed since the administering the second dose, wherein the third dose is about 400 mCi or higher and the second duration is at least a week.

7. The method of claim 6, wherein the third dose is about 500 mCi.

8. The method of claim 6, wherein the administering the first dose and the administering the second dose are performed by intravenous infusion over a duration of from 3 to 6 hours.

9. The method of claim 6, wherein the second duration is from 6 to 16 weeks.

10. The method of claim 6, wherein the second duration is from 10 to 12 weeks.

11. A composition for treating a neuroendocrine tumor, comprising ¹¹¹In-pentetreotide having a radioactivity of about 400 mCi or higher in a single dose.

12. The composition of claim 11, wherein the radioactivity is about 500 mCi.

13. The composition of claim 11, wherein the ¹¹¹In-pentetreotide is substantially free of cadmium, iron, lead, zinc, aluminum and copper.
